(19) United States
(12) Patent Application Publication
(10) Pub. No.: US 2023/0256052 A1
(43) Pub. Date: Aug. 17, 2023

(54) METHOD AND COMPOSITION FOR REDUCING OR PREVENTING BONE RESORPTION

(71) Applicant: University of Connecticut, Farmington, CT (US)

(72) Inventors: Tannin Avery Schmidt, Farmington, CT (US); Joseph Lorenzo, Farmington, CT (US); Sun-Kyeong Lee, Farmington, CT (US); Adam Tanguay, Farmington, CT (US)

(21) Appl. No.: 18/012,288

(22) PCT Filed: Jul. 7, 2021
(86) PCT No.: PCT/US2021/040661

§ 371 Date: Dec. 22, 2022

Related U.S. Application Data
(60) Provisional application No. 63/048,840, filed on Jul. 7, 2020.

(51) Int. Cl. A61K 38/17; A61P 19/10; A61K 47/69

(52) U.S. Cl. CPC A61K 38/1709; A61P 19/10; A61K 47/6903

(57) ABSTRACT

An anti-osteoclastic composition containing a proteoglycan 4 in a pharmaceutically acceptable carrier is administered to a human or animal suffering from a pathological bone condition such as osteoporosis in a sufficient amount to reduce, delay onset of or prevent excessive bone resorption in which the rate of bone resorption exceed the rate of bone formation. Excessive bone resorption is inhibited by preventing the formation or activity of osteoclasts. Osteoporosis is linked to bone fracture, a debilitation condition often resulting in the need for risky, invasive surgery, and the possible consequence of permanent immobilization, especially in elderly populations.

### CROSS REFERENCE TO RELATED APPLICATION

This application claims priority to U.S. Provisional Application No. 63/048,840, filed Jul. 7, 2020, which is hereby incorporated by reference in its entirety.

### FIELD OF THE INVENTION

The present application relates to the field of osteoporosis and particularly relates to compositions containing recombinant proteins encoded by the proteoglycan 4 gene and use of those compositions to reduce or prevent bone resorption.

### BACKGROUND

Osteoporosis is a disease caused by low bone mass and structural deficiencies in bone. It develops when the rates of bone resorption, mediated by osteoclasts, exceed rates of bone formation, mediated by osteoblasts. The most damaging consequence of osteoporosis is bone fracture. An estimated 2-7 million hip fractures occurred in 2010 worldwide, of which 1,364,717 (51%) were calculated to be potentially preventable (264,162 in men, and 1,100,555 in women), if osteoporosis (defined as a femoral neck T score, as measured by dual energy absorptiometry, of −2·5 SD or less) could be avoided.

Several therapies are currently available for the treatment of osteoporosis. However, all have significant risks (1). Bisphosphonates and denosumab, are associated with the adverse outcomes of osteonecrosis of the jaw and atypical subtrochanteric fractures (2, 3). Although the exact etiology of these complications is unknown, there is concern that they may result from coupled decreases in the rates of bone resorption and bone formation. Safety concerns with teriparatide, a PTH analog, which stimulates bone turnover and a net gain of bone, limit its use to 24 months (4). Odanacatib (5), a cathepsin K inhibitor, decreases resorption without affecting formation. It was considered for development as a therapy for osteoporosis, but was abandoned when it was found to produce an increased risk of cerebral vascular accidents. Romosozumab, an anti-sclerostin antibody, was recently approved for use in patients as an anabolic. However, it has been associated with an increased risk of cardiovascular events (6). Abaloparatide (7), a PTHrP analog, is similar to teriparatide in its actions and, like teriparatide, its administration is limited to a maximum of 24 months because of safety concerns.

The risks and benefits of the approved therapies for osteoporosis have been documented by numerous rigorous clinical trials (1). However, because of concerns by patients about side effects, their use has not been as widespread as they might be (1). Hence, there is a great need to identify additional therapies for treating inflammatory bone diseases that have strong efficacy and a reduced likelihood of untoward effects compared to existing therapies.

Current drugs used to treat enhanced osteoclast activity and bone loss of cancer, inflammatory arthritis and osteoporosis are effective, but have significant adverse effects, which limit their acceptance by the patients who could most benefit from them.

Therefore, compositions are needed for the treatment and prevention of osteoporosis that are easily produced and, when administered to humans and animals who exhibit excessive bone resorption or to whom will be administered other drugs that could cause excessive bone resorption, result in reduced or normal bone resorption, thereby avoiding or reversing osteopenia and/or osteoporosis.

### SUMMARY

Described herein are anti-osteoclastic compositions and methods for reducing or preventing bone resorption. Excessive bone resorption is a cause of osteopenia and osteoporosis when the rate of bone resorption exceed the rate of bone formation. The anti-osteoclastic compositions include recombinant human proteoglycan 4 (rhPRG4), which unexpectedly inhibits osteoclast formation and function when sufficient amounts are administered. In accordance with the method, the anti-osteoclastic compositions described herein, containing rhPRG4, are administered to an individual in a sufficient amount to reduce or prevent excessive bone resorption, thereby minimizing or delaying onset or reducing osteopenia or, in more severe cases of bone resorption, minimizing onset or reducing osteoporosis. The method is useful for preventing or slowing the onset of bone loss in a number of pathological conditions, or simply through the natural aging process.

The method described herein also includes administration before, after or in combination with a second anti-osteoclastic composition. The compositions are administered via any of several routes of administration, including orally, parenterally, intravenously, intraperitoneally, intracranially, intraspinally, intrathecally, intraventricularly, intramuscularly, subcutaneously, intracavity, transdermally, or locally either directly or via delivery (potentially delayed) of a hydrogel or appropriate biomaterial, such as a DNA-inspired nanomaterial. Pharmaceutical compositions can also be delivered locally to the area in need of treatment, for example by topical application or local injection. Effective doses for any of the administration methods described herein can be extrapolated from dose-response curves derived from in vitro or animal model test systems.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present document describes and refers to various embodiments of the invention. No particular embodiment is intended to define the scope of the invention. Rather, the embodiments merely provide non-limiting examples of various methods, that are at least included within the scope of the invention. Some embodiments of the present invention are summarized below, while others are described and shown elsewhere in the present document.

### BRIEF DESCRIPTION OF THE DRAWINGS

Features and advantages of the methods of the present invention will be more fully appreciated by reference to the following detailed description of presently preferred but nonetheless illustrative embodiments in accordance with the present invention when taken in conjunction with the accompanying drawings in which:

FIG. 1 is a visual reproduction of an SDS-PAGE gel showing rhPRG4 visualized as bands at and above ˜460 kDa.

FIG. 2 is a visual reproduction of an SDS-PAGE gel stained with Coomassie blue showing that CTSK is capable of enzymatic digestion of rhPRG4.

FIG. 3 is a graph showing inhibition of CTSK activity by rhPRG4.

FIG. 4 is a bar graph showing inhibition of in vitro osteoclastogenesis by rhPRG4.

FIG. 5 is a bar graph showing inhibition of in vitro osteoclastogenesis by rhPRG4.

FIGS. 6A and 6B are bar graphs showing inhibition of in vitro osteoclast activity

FIGS. 7A and 7B show the binding interaction of RANK with 300 ng/ml rhPRG4 showing that rhPRG4 does not bind RANK.

FIGS. 8A and 8B show the in vitro effect of rhPRG4 on RANK and RANKL binding showing that rhPRG4 inhibits the binding interaction of RANK and RANKL.

FIGS. 9A and 9B show the in vitro effect of rhPRG4 on RANK and RANKL binding showing that rhPRG4 may possibly inhibit the binding interaction of RANK and RANKL.

FIGS. 10A and 10B are graphs showing rhPRG4's inhibition of in vivo osteoclastogenesis and potential osteoclast activity in an inflammatory bone loss model.

FIGS. 11A-11F show several bar graphs showing femurs of PRG4 deficient mice have is several graphs showing femurs of PRG4 deficient mice have diminished mechanical properties compared to PRG4 deficient mice who've had global expression of PRG4 turned back via injection of tamoxifen.

FIG. 12 shows the strength of binding between RANKL and rhPRG4.

FIG. 13 shows the strength of binding between TNFR1 and rhPRG4.

FIGS. 14A and 14B show the in vitro effect of rhPRG4 on RANK and RANKL binding showing that rhPRG4 inhibits the binding interaction of RANK and RANKL.

FIG. 15A is a graph showing the effect of PRG4 on RANK/RANKL binding.

FIG. 15B is a graph showing the effect of fibronectin on RANK/RANKL binding.

FIGS. 16A and 16B show the in vitro effect of rhPRG4 on TNFa/TNFR1 binding showing that rhPRG4 inhibits the binding interaction of TNFa and TNFR1.

FIGS. 17A and 17B is a graph showing the effect of PRG4 on RANK/RANKL binding.

FIGS. 18A, 18B and 18C is a graph showing the effect of PRG4 on TNFa binding.

### DETAILED DESCRIPTION

Compositions and methods for reducing or preventing bone resorption are provided. Excessive bone resorption is a cause of osteopenia and osteoporosis when the rate of bone resorption exceed the rate of bone formation. Osteoporosis is linked to bone fracture, a debilitation condition often resulting in the need for risky, invasive surgery, and the possible consequence of permanent immobilization, especially in elderly populations.

The anti-osteoclastic compositions described herein include recombinant human proteoglycan 4 (rhPRG4). Applicants unexpectedly discovered that PRG4 (Lubricin) represents a new class of anti-osteoclastic compounds, which inhibit osteoclast formation and function. Using a variety of assays, applicants have demonstrated both in vitro and in vivo that osteoclastogenesis and osteoclast activity (bone resorption) is inhibited by administration of sufficient amounts of PRG4. The role that endogenous PRG4 expression plays in bone microstructure and mechanical properties was also demonstrated and is described below.

In accordance with the method, the anti-osteoclastic compositions described herein, containing rhPRG4, are administered to an individual in a sufficient amount to reduce or prevent excessive bone resorption, thereby minimizing or delaying onset or reducing osteopenia or, in more severe cases of bone resorption, minimizing onset or reducing osteoporosis. The method is useful for preventing or slowing the onset of bone loss in a number of pathological conditions, or simply through the natural aging process.

The method described herein also includes administration before, after or in combination with a second anti-osteoclastic composition. Such a combination therapy may increase the bioactivity of the second anti-osteoclastic composition so that the second anti-osteoclastic composition can be administered less frequently or at a lower concentration.

It was surprisingly discovered that administration of PRG4 acts on osteoclasts and prevents their formation or activity.

The proteoglycan 4 gene (PRG4) encodes megakaryocyte stimulating factor (MSF) as well as highly glycosylated differently splice variant and glycoforms of a “superficial zone protein” also known as lubricin. Superficial zone protein was first localized at the surface of explant cartilage from the superficial zone and identified in conditioned medium. Lubricin was first isolated from synovial fluid and exhibited lubricating ability in vitro similar to synovial fluid at a cartilage-glass interface and in a latex-glass interface. It was later identified as a product of synovial fibroblasts, and its lubricating ability was discovered to be dependent on O-linked β (1-3) Gal-GaINAc oligosaccharides within a large mucin like domain of 940 amino acids encoded by exon 6. Lubricin molecules are differentially glycosylated and several naturally occurring splice variants have been reported. They are collectively referred to herein as PRG4. PRG4 has been shown to be present inside the body at the surface of synovium, tendon, articular cartilage such as meniscus, and in the protective film of the eye, among other sites, and plays an important role in joint lubrication and synovial homeostasis.

Prior to the discovery described herein, PRG4 had been appreciated as a protein with only mechanical properties, providing mechanical functionalities such as lubricating joints, tendons, cartilage, and acting as a mechanical barrier to inhibit intercellular interactions.

Recently, lubricin has been shown to possess properties that extend beyond its ability to provide boundary lubrication and anti-adhesion. (WO2016123123A1) In particular, PRG4 has been shown to possess anti-inflammatory properties due to its ability to act as a ligand or signaling molecule, participating in ligand receptor interactions to modulate, for example, CD44 activation, NF-κB translocation, and cytokine-mediated inflammation. However, prior to the discoveries described herein, administration of PRG4 for the treatment or prevention of bone resorption was not previously contemplated.

Cathepsin K (CTSK) is a papain-like cysteine protease member of the cathepsin family of lysosomal proteases and is the only cathepsin expressed at high levels in osteoclasts. CTSK is the primary enzyme responsible for degradation of type I collagen, which composes ˜90% of the bone organize matrix. CTSK inhibitors have been in development as treatment for osteoporosis, however clinical trials have been terminated due to unforeseen side effects.

It is shown in the examples provide below that PRG4 itself is also degraded by CTSK, potentially serving as an alternative substrate in bone and, therefore, plays a role in regulating bone resorption.

When combined with a pharmaceutically acceptable carrier, including diluents and the like, the rhPRG4 protein provides the anti-osteoclastic composition described herein, which is useful, when administered to a human or animal in an effective amount, for reducing or preventing excessive bone resorption, thus providing an anti-osteoclastic composition for the treatment, reduction, inhibition, delay or prevention of osteopenia, osteoporosis, and/or bone resorption associated with cancer or adverse effects of cancer treatment, such as, but not limited to breast cancer metastasis to bone.

A variety of diseases or conditions can increase bone loss such as rheumatoid arthritis and other rheumatological conditions, malabsorption syndromes, sex hormone deficiency (hypogonadism), primary hyperparathyroidism, chronic kidney disease, chronic liver disease, diabetes, chronic obstructive pulmonary disease (COPD), untreated hyperthyroidism, and neurological disorders.

An inflammatory disease of the joints, rheumatoid arthritis is often treated with glucocorticoids, usually prednisone. Pain and loss of joint function can lead to inactivity, which can further contribute to bone loss. Research suggests that osteoclast (a bone removing cell) activity and bone resorption is increased at the affected sites. In addition to rheumatoid arthritis, ankylosing spondylitis has been associated with bone loss. Several other rheumatological conditions may affect the joints, resulting in poor balance and increased risk of falls, including lupus, psoriatic arthritis and severe osteoarthritis of the hip or knee.

Malabsorption can result from bowel diseases such as Crohn's disease, ulcerative colitis and celiac disease, and other conditions that affect the bowel such as weight loss surgery. These conditions reduce the absorption of nutrients from the intestine including dietary calcium and vitamin D. The result is lower levels of calcium and vitamin D, which can increase bone loss and falls risk, leading to fractures.

In women, sex hormone deficiency generally results in the early stoppage of menstrual periods (amenorrhea). Common causes include premature menopause (before the age of 45), eating disorders such as anorexia nervosa, exercise-induced amenorrhea (typically seen in high performance athletes and dancers), pituitary disease, chemotherapy and chronic illness. Some of these conditions can be treated with hormone therapy.

In men, low levels of testosterone can be caused by a number of conditions including liver disease, pituitary disease, chemotherapy, chronic illness and aging. Some of these conditions can be treated with testosterone.

The parathyroid glands produce parathyroid hormone, which controls blood calcium levels. In primary hyperparathyroidism a tumor (generally benign) in one or more of these glands causes the production of more parathyroid hormone than is needed. This causes an increase in bone turnover, which results in excess calcium release from bone and a rise in the level of calcium in the blood. As a result, the risk of osteoporosis and fractures also increases.

Many patients with chronic kidney disease are treated with glucocorticoids such as prednisone, which puts them at risk for developing osteoporosis. In addition, chronic kidney disease may cause several different metabolic bone diseases (called renal osteodystrophy) that are associated with reduced bone formation, hyperparathyroidism, and vitamin D deficiency. In renal osteodystrophy, bone quality is poor.

Chronic liver disease is associated with reduced bone formation, vitamin D deficiency and low sex hormones, all of which may result in bone loss. In addition, some forms of liver disease may be treated with glucocorticoids such as prednisone, which may cause even greater bone loss. Up to 50% of patients with chronic liver disease develop osteoporosis.

There is evidence to suggest that both men and women with type 1 diabetes are at higher risk for low bone density and for osteoporotic fractures. Poorly controlled type I and type II diabetes are often associated with hypoglycemic episodes (low blood sugar) and/or neuropathy (poor sensation) in the feet. Both of these complications of diabetes can increase the risk of falls and fractures.

COPD is a type of chronic lung disease that usually results after prolonged smoking but can also occur due to other causes. COPD can consist of chronic bronchitis or emphysema or both, and is often associated with a chronic cough, phlegm production, shortness of breath on exertion or at rest (depending on the severity) and frequent chest infections. There is a strong association between COPD and low bone mass or osteoporosis, usually from a combination of factors such as smoking history, low body weight, poor nutrition and treatment with oral glucocorticoids.

Normal thyroid hormone levels maintain good bone health. Too much thyroid hormone interferes with the body's ability to absorb calcium into the bones and increases bone turnover, which can cause bone loss over time.

Several neurological disorders are associated with an increased risk of bone loss, particularly conditions or injuries resulting in immobility. This includes stroke, multiple sclerosis and spinal cord injury.

Other conditions or diseases that may increase bone loss include Cushing's syndrome, Pituitary disease, Multiple myeloma, Thalassemia major, and AIDS/HIV. (14)

### Dosages, Formulations and Administration

The term effective amount, as used throughout, is defined as any amount, for example, an amount of composition, necessary to produce one or more desired effect, such as treatment, reduction, delay or prevention of bone resorption. For example, the dosage is optionally less than about 10 mg/kg and can be less than about 9.5, 9, 8.5, 8, 7.5, 7, 6.5, 6, 5.5, 5, 4.5, 4, 3.5, 3, 2.5, 2, 1.5, 1.25, 1.0, 0.9, 0.8, 0.7, 0.6, 0.5, 0.4, 0.3, 0.2, 0.1, 0.01, 0.001, 0.0001 mg/kg or any dosage in between these amounts. The terms “about” or “approximately” are used herein to indicate that a value includes the inherent variation of error for the device, the method being employed to determine the value, or simply error-tolerance of a value. For example, the terms “about” or “approximately” may mean±1%, ±5%, ±10%, ±15% or ±20% variation from a predetermined value. The dosage can range from about 0.1 mg/kg to about 10 mg/kg, from about 0.1 mg/kg to about 9 mg/kg, from about 0.1 mg/kg to about 8 mg/kg, from about 0.1 mg/kg to about 7 mg/kg, from about 0.1 mg/kg to about 6 mg/kg, from about 0.1 mg/kg to about 5 mg/kg, from about 0.1 mg/kg to about 4 mg/kg, from about 0.1 mg/kg to about 3 mg/kg, from about 0.1 mg/kg to about 2.5 mg·kg, from about 0.1 mg/kg to about 2 mg/kg, from about 0.1 mg/kg to about 1.5 mg/kg, from about 0.1 mg/kg to about 1 mg/kg, or from about 0.1 mg/kg to about 0.5 mg/kg. The dosages can be adjusted based on specific characteristics of the anti-osteoclastic composition and the subject receiving it.

Effective amounts and schedules for administering the anti-osteoclastic composition provided herein can be determined empirically. The dosage ranges for administration are those large enough to produce the desired effect in which one or more symptoms of the disease or condition are affected (for example, inhibited, reduced or delayed or prevented). The dosage should not be so large as to cause substantial adverse side effects, such as unwanted cross-reactions, unwanted cell death, and the like. Generally, the dosage will vary with the age, body weight, general health, sex and diet of the subject, the mode and time of administration, rate of excretion, drug combination, and severity of the particular condition. The dosage can be adjusted by the individual physician in the event of any contraindications. Dosages can vary, and can be administered in one or more dose administrations daily.

The anti-osteoclastic composition used in the methods according to the embodiments of the present invention can be provided in a pharmaceutical composition. These include, for example, a pharmaceutical composition containing a therapeutically effective amount of one or more of the anti-osteoclastic compositions and a pharmaceutical carrier. The term carrier means a compound, composition, substance, or structure that, when in combination with a compound or composition, aids or facilitates preparation, stability, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers or excipients, including, but not limited to, saline, buffered saline, dextrose, and water.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in various sources and manuals. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain agents such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included.

Solid dosage forms for oral administration of the anti-osteoclastic composition include capsules, tablets, pills, powders, and granules. In such solid dosage forms, the active ingredient described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also include buffering agents.

Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients. For example, local sustained or delayed delivery of the composition can be accomplished by administering, applying directly or even wrapping the area experiencing or susceptible to bone resorption, such as a bone, with a hydrogel containing the composition.

Liquid dosage forms for oral administration of the anti-osteoclastic composition include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

When used in the methods according to the embodiments of the present invention, the anti-osteoclastic composition can be administered in a number of ways depending on whether local or systemic treatment is desired, and on the area to be treated. The compositions are administered via any of several routes of administration, including orally, parenterally, intravenously, intraperitoneally, intracranially, intraspinally, intrathecally, intraventricularly, intramuscularly, subcutaneously, intracavity, transdermally, or locally either directly or via delivery (potentially delayed) of a hydrogel or appropriate biomaterial, such as a DNA-inspired nanomaterial as described in published US patent application No. US 2017/0362238 A1. Pharmaceutical compositions can also be delivered locally to the area in need of treatment, for example by topical application or local injection. Effective doses for any of the administration methods described herein can be extrapolated from dose-response curves derived from in vitro or animal model test systems.

Throughout, treat, treating, and treatment refer to a method of reducing, inhibiting, preventing or delaying bone resorption or one or more effects or symptoms of excessive bone resorption. The effect of the administration to the subject can have the effect of but is not limited to reducing one or more symptoms associated with excessive bone resorption. The effect of the administration to the subject can have the effect of but is not limited to reducing the speed or amount of bone resorption. For example, a disclosed method is considered to be a treatment if there is about a 5% reduction in bone resorption when compared to the subject prior to treatment or when compared to a control subject or control value. Thus, the reduction can be about a 5, 10, 20, 30, 40, 50, 60, 70, 80, 90, 100%, or any amount of reduction in between.

As utilized herein, by prevent, preventing, or prevention is meant a method of precluding, delaying, averting, obviating, forestalling, stopping, or hindering the onset, incidence, severity, or recurrence of excessive bone resorption. For example, the disclosed method is considered to be a prevention if there is a reduction or delay in onset, incidence, severity, or recurrence of excessive bone resorption or associated conditions in a subject susceptible to osteopenia or osteoporosis as compared to control subjects susceptible to osteopenia or osteoporosis that did not receive the anti-osteoclastic composition.

### rhPRG4 Production

PRG4 is described and methods of isolating, purifying or recombinantly expressing PRG4 are described in US Patent Application Publication No. US 2018/0015141 A1, the entire contents of which is incorporated by reference herein. Methods of producing rhPRG4, are described in US Patent Application Publication No. US 2019/0270783 A1, the entire contents of which is incorporated by reference herein.

The following non-limiting examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLES

### Example 1: Degradation of PRG4 by CTSK

Cathepsin K (CTSK) is secreted by osteoclasts to degrade collagen and other matrix proteins during bone resorption. CTSK is a papain-like cysteine protease member of the cathepsin family of lysosomal proteases and is the only cathepsin expressed at high levels in osteoclasts. CTSK is the primary enzyme responsible for degradation of type I collagen, which composes approximately 90% of the bone organize matrix. CTSK inhibitors have been in development as a potential treatment for osteoporosis, however clinical trials were terminated due to adverse side effects.

The results of this experiment demonstrate that PRG4 itself is also degraded by CTSK, potentially serving as an alternative substrate in bone and therefore plays a role in regulating bone resorption.

### Materials and Methods

In a first experiment, 1.5 μg rhPRG4 was co-incubated with 30 ng CTSK for 2, 6, or 24 hours. SDS-PAGE was used to assess enzymatic digestion by CTSK and performed as previously described (PMID 23257245 (15)). Briefly, samples were loaded onto a Novex 3-8% Tris-Acetate gel in NuPAGE Tris-Acetate SDS running buffer and electrophoresed at 150 V for 75 minutes. After, the gel was stained using Coomassie G-250 (Simplyblue SafeStain) to visualize proteins per manufacturer's instructions.

In a second experiment, 1.5 μg rhPRG4 was co-incubated with 7.5 (0.5%), 15 (1.0%), or 30 ng (2.0%) CTSK for 0, 1, 2, or 6 hours. SDS-PAGE to assess enzymatic digestion by CTSK was performed as described above.

In a third experiment, ProCTSK was purchased from Enzo Life Sciences (#BML-SE367-0010) and activated using 32.5 mM, pH 3.5 sodium acetate through co-incubation for 30 minutes at room temperature. 50 ng, 20 ng, and 5 ng of activated CTSK were each incubated with 1 ng, 10 ng, and 100 ng of rhPRG4, in addition to CTSK substrate Ac-LR-AFC provided in the activity assay kit (200 μM). Samples were incubated in a 96 well half area opaque plate for 2 hours, with reads at 400 nm excitation and 505 nm emission wavelengths at T=0, 1, and 2 hours. When CTSK cleaves the Ac-LR-AFC substrate, it results in signal detection using the excitation/emission wavelengths above.

### Results/Analysis

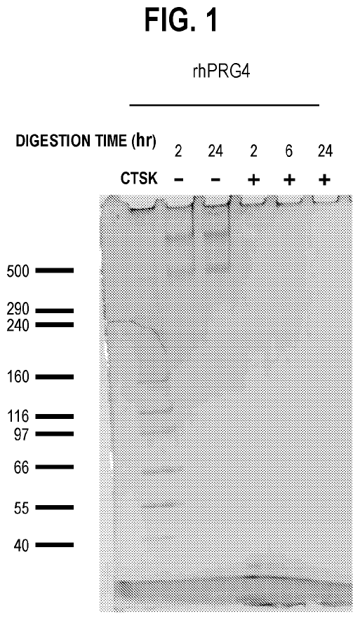

The results set forth in FIG. 1 show rhPRG4 visualized as the monomeric band at ˜460 kDa and the dimeric band above, in a 3-8% SDS-PAGE gel stained with Simply Blue Coomassie total protein stain, in the absence of CTSK. When incubated with CTSK at 2, 6, or 24 hours, rhPRG4 is detected at <40 kDa. This indicates enzymatic digestion of rhPRG4 by CTSK. Band intensity of rhPRG4+CTSK decreases with time, indicating continued enzymatic digestion over longer time periods.

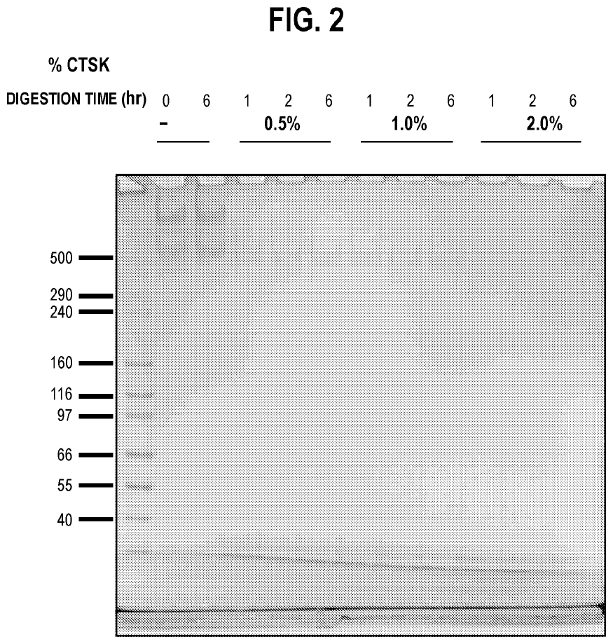

A second 3-8% SDS-PAGE gel stained with Simply Blue Coomassie total protein stain is set forth in FIG. 2, which provided additional evidence that CTSK is capable of enzymatic digestion of rhPRG4. A greater amount of CTSK enzyme results in increased enzymatic digestion, indicated by the changes in band intensities.

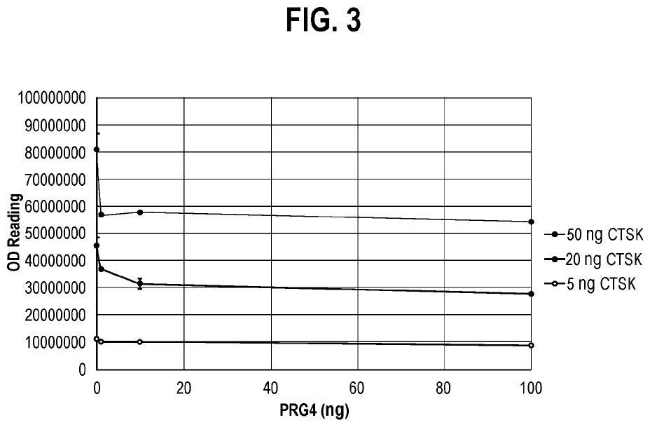

Motivated by the results presented in FIGS. 1 and 2, FIG. 3 shows rhPRG4 inhibits CTSK digestion of a preferred substrate, by acting as a substrate itself. Using a commercially available Cathepsin-K Activity Assay kit, which is a fluorescence-based assay that utilizes the preferred cathepsin-K substrate sequence LR labeled with AFC (amino-4-trifluoromethyl coumarin), samples CTSK will cleave the synthetic substrate LR-AFC to release free AFC. The released AFC can easily be quantified using a fluorometer or fluorescence plate reader. FIG. 3 shows that a greater amount of CTSK increases the amount of substrate cleaved, resulting in an increase in signal. Addition of rhPRG4 resulted in decreased signal, indicating that rhPRG4 may be acting as a competitive substrate for CTSK.

### Example 2: Inhibition of In Vitro Osteoclastogenesis and Osteoclast Activity by PRG4

In this experiment, the ability of PRG4 to inhibit in vitro osteoclastogenesis and osteoclast activity was examined.

### Materials and Methods

In a first study, wild type (WT) murine bone marrow macrophages were cultured with M-CSF, RANKL, and rhPRG4 or vehicle for 5 days. A stock sample of rhPRG4 (1.33 mg/ml stock) was diluted to 1/10, 1/30, or 1/100 concentration.

In a second study, WT murine bone marrow macrophages were allowed to attach to the plate for 3 days before adding M-CSF, RANKL, and rhPRG4. Cells were then incubated for an additional 3 days.

In a third study, WT murine bone marrow macrophages were cultured on pieces of bone with M-CSF and RANKL for 14 days. Group 1 received vehicle control (PBS). Group 2 received PRG4 at 133 ug/ml on day 7 through day 14. Group 3 received rhPRG4 at 133 ug/ml on day 0 through day 14.

In each of the above experiments, osteoclast differentiation and activity were evaluated as previously described (8). Briefly, at the end of the experiment (as described above) cells were fixed in 2.5% glutaraldehyde, followed by staining with tartrate resistant acid phosphatase (TRAP). TRAP-positive cells with three or more nuclei were considered osteoclasts. Area of tracts resorbed on the pieces of bone by motile cells and area of the pits formed by non-motile cells were imaged, and used as measures of in vitro osteoclast activity.

### Results/Analysis

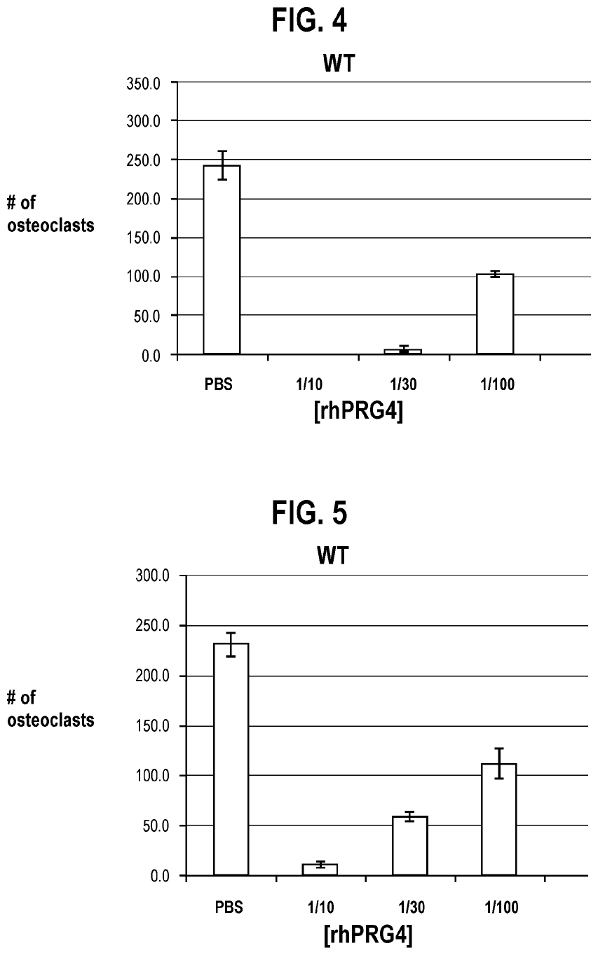

The results of the first study are shown in FIG. 4 in which increased concentrations of rhPRG4 resulted in a dose dependent decrease in osteoclast differentiation. As the PRG4 was added at the same time as the cells (day 0 of culture), M-CSF, and RANKL, it is possible that the rhPRG4 influenced differentiation by inhibiting adherence of the bone marrow macrophages to the plate.

The results of the second study are shown in FIG. 5. As indicated, rhPRG4 still inhibited osteoclast differentiation when introduced after the cells had adhered to the plate, though to a slightly lesser extent than the previous experiment.

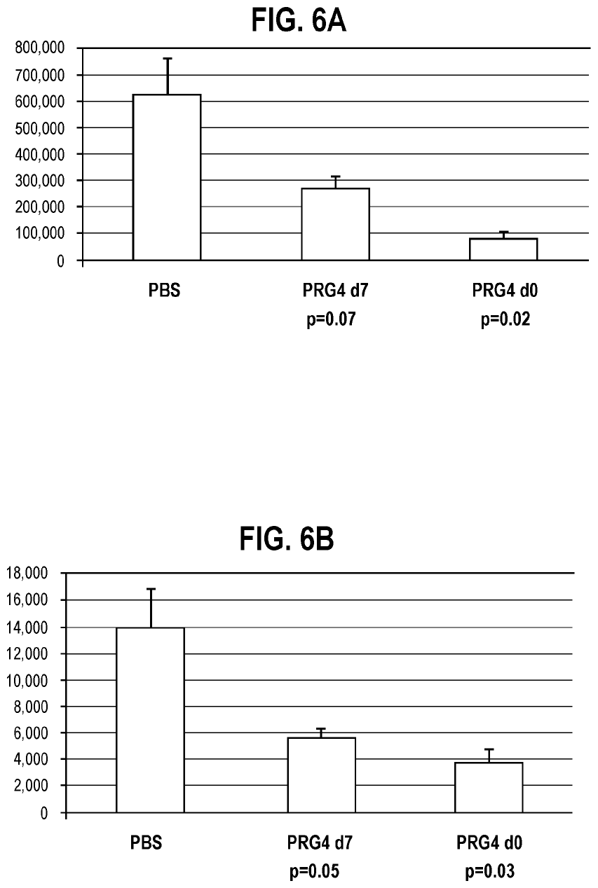

The results of the third study, to assess in vitro osteoclast activity, are shown in FIG. 6. Both groups that received rhPRG4 had a decrease in bone resorption, with the effect approaching statistical significance in Group 2 (which received the rhPRG4 on day 7 of a 14 day culture) and statistically significant in Group 3 (which received the rhPRG4 on day 0 of a 14 day culture.

### Example 3: Binding of PRG4 to RANK

In this experiment, the ability of PRG4 to bind RANK was examined. The binding of RANK to RANKL is a critical step in osteoclast precursor maturation. PRG4's ability to inhibit osteoclastogenesis and activity may be due to its ability to interfere with this interaction.

### Materials and Methods

In a first study, to test the ability of PRG4 to RANK, biotinylated rhPRG4 (bPRG4) was bound to a streptavidin-conjugated donor bead, using the AlphaLISA platform beads. His-tagged RANK (Sino Biological, 16078-H08H) was bound to a Ni-conjugated acceptor bead. If bPRG4 and his-RANK bind, the donor and acceptor beads will be come in close proximity of one another, generating an increase in signal. rhPRG4 was added at 300 ng/ml, and RANK was added in combination at 0.3, 1, 3, 10, and 30 ng/ml. All sample proteins and beads were diluted in PBS+0.1% BSA. Both the streptavidin donor beads and the nickel chelate acceptor beads were included in each sample at 20,000 ng/ml. bPRG4 and hisRANK were added to wells in a 96 well half area opaque plate and allowed to interact for 2 hours with shaking at 150 rpm. The acceptor beads were then added for an hour with shaking, followed by the donor beads for another hour with shaking. Samples were read using a Spectramax i3x plate reader with an excitation wavelength of 680 nm and emission of 570 nm.

In a second study, biotinylated RANKL (bRANKL) was bound to a streptavidin-conjugated donor bead. His-tagged RANK was bound to a Ni-conjugated acceptor bead. When RANK binds RANKL, the donor and acceptor beads are brought into close proximity of one another and signal is generated. PRG4 was introduced to assess its ability to inhibit this interaction. rhPRG4 was added at 30, 300, 3000, and 10000 ng/ml, while hisRANK and bRANKL were added at 30 and 300 ng/ml in combination with the rhPRG4. All sample proteins and beads were diluted in PBS+0.1% BSA. Both the streptavidin donor beads and the nickel chelate acceptor beads were included in each sample at 20,000 ng/ml. rhPRG4 and bRANKL were added to wells in a 96 well half area opaque plate and allowed to interact for 2 hours with shaking at 150 rpm. HisRANK was then added for an hour with shaking. After that, the acceptor beads were added for an hour with shaking, followed by the donor beads for another hour with shaking. Samples were read using a Spectramax i3x plate reader with an excitation wavelength of 680 nm and emission of 570 nm.

In a third study, biotinylated RANKL (bRANKL) was bound to a streptavidin-conjugated donor bead. His-tagged RANK was bound to a Ni-conjugated acceptor bead. When RANK binds RANKL, the donor and acceptor beads are brought into close proximity of one another and signal is generated. PRG4 was introduced, along with a non-specific glycoprotein control (Fibronectin) to assess their ability to inhibit this interaction. rhPRG4 (or fibronectin) was added at 30, 300, 3000, and 10000 ng/ml, while hisRANK and bRANKL were added at 30 ng/ml each in combination with the rhPRG4. All sample proteins and beads were diluted in PBS+0.1% BSA. Both the streptavidin donor beads and the nickel chelate acceptor beads were included in each sample at 20,000 ng/ml. rhPRG4 (or fibronectin) and bRANKL were added to wells in a 96 well half area opaque plate and allowed to interact for 2 hours with shaking at 150 rpm. HisRANK was then added for an hour with shaking. After that, the acceptor beads were added for an hour with shaking, followed by the donor beads for another hour with shaking. Samples were read using a Spectramax i3x plate reader with an excitation wavelength of 680 nm and emission of 570 nm.

### Results/Analysis

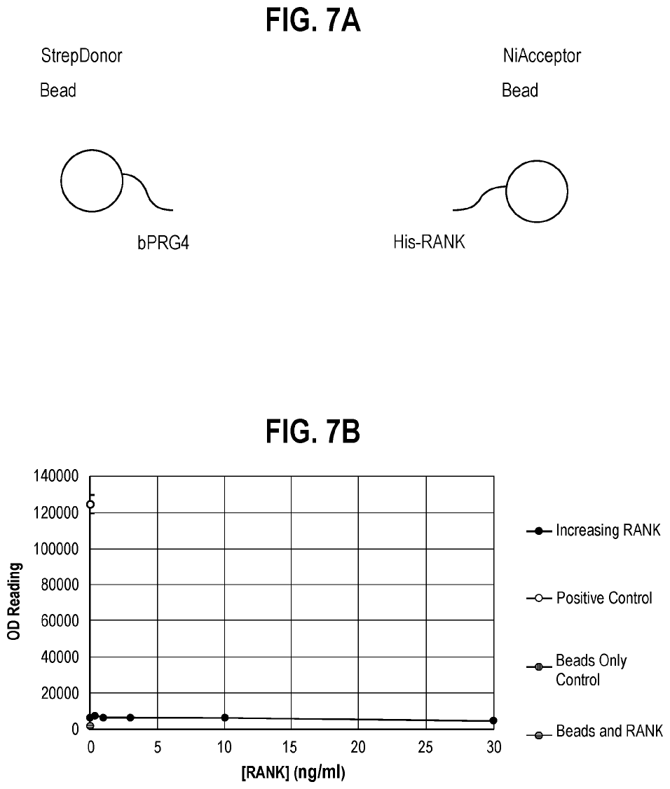

As shown in FIG. 7, signal did not increase over the negative control. This suggests PRG4 does NOT bind to RANK.

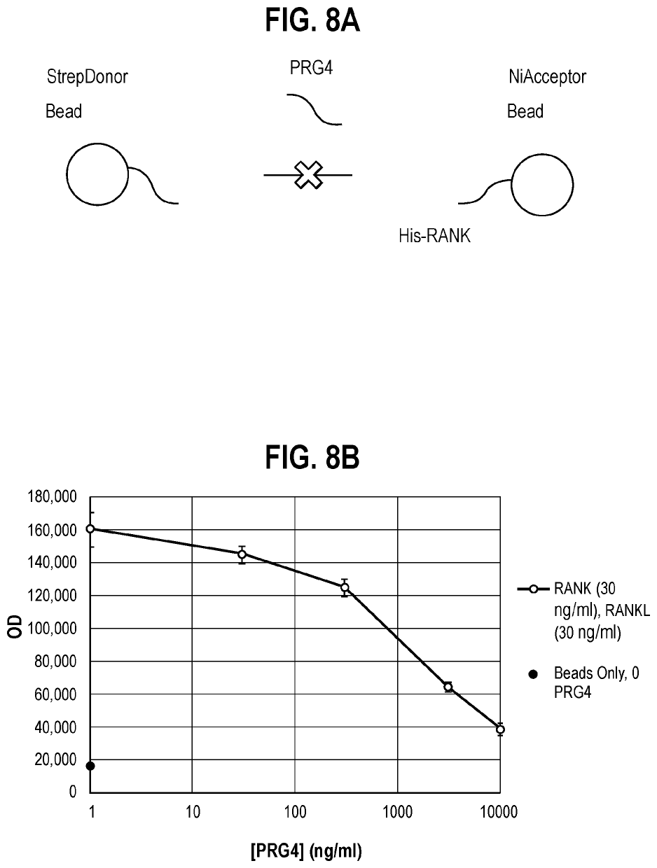

As shown in FIG. 8, addition of rhPRG4 to RANKL, prior to the addition of RANK, dose-dependently decreased the signal. One plausible mechanism for this disruption would be rhPRG4 binding to RANKL, preventing RANKL from binding RANK. As noted previously, PRG4 did not bind RANK.

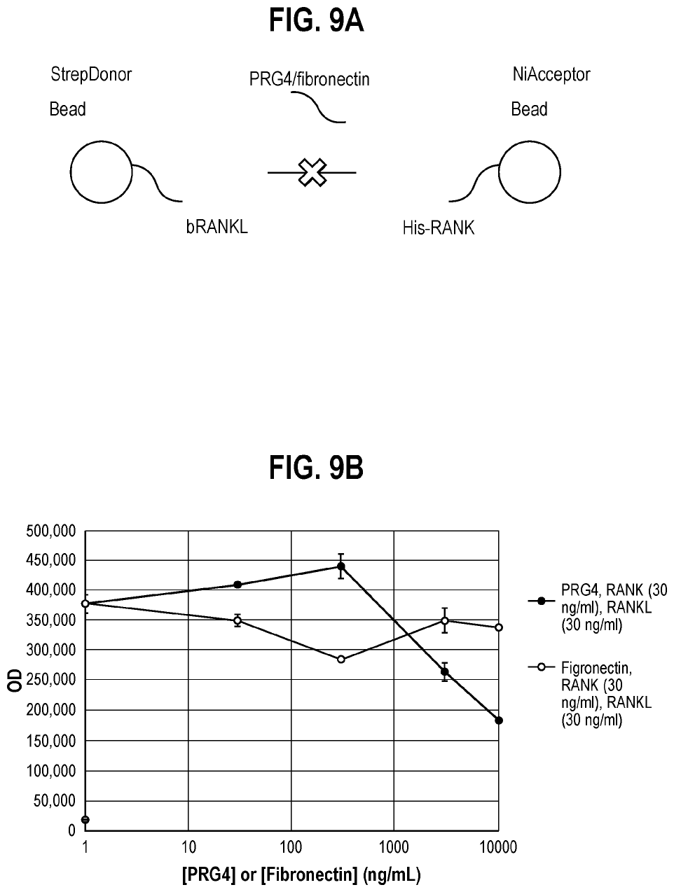

As shown in FIG. 9, addition of bPRG4, as in FIG. 8 with RANKL prior to addition of RANK, again resulted in decreased signal at the two highest concentrations. To establish that the inhibitory effect of rhPRG4 was not due to non-specific interactions, fibronectin, a glycoprotein similar in molecular weight to PRG4, was tested as well. Addition of fibronectin did not lead to a decrease in signal at the two highest concentrations. This demonstrates PRG4's inhibition of the RANKL-RANK is not due to non-specific interactions.

### Example 4: Effect of PRG4 on TNFα

In this experiment, the calvarial TNFα inflammatory model of Jastrzebski S et al. (8) was used to quickly induce significant osteoclast differentiation in the calvaria of mice and the effect of PRG4 on the osteoclast differentiation was studied.

### Materials and Methods

2 ug TNFa (approximately 100 ug/kg) was injected over the calvaria for 4 days in 7 to 8 week old C57BL/6 male mice (N=5 per group). Mice also received daily IP injections (0.25 ml) of either rhPRG4 at 1.33 mg/ml IP or vehicle control (PBS+0.01% Tween20). After 5 days mice were sacrificed and calvaria were embedded in paraffin. After, calvaria were sectioned (8 μm slices) and TRAP stained in order to stain for osteoclasts. Osteomeasure software was used to perform histomorphometric analysis as described previously (8).

### Results/Analysis

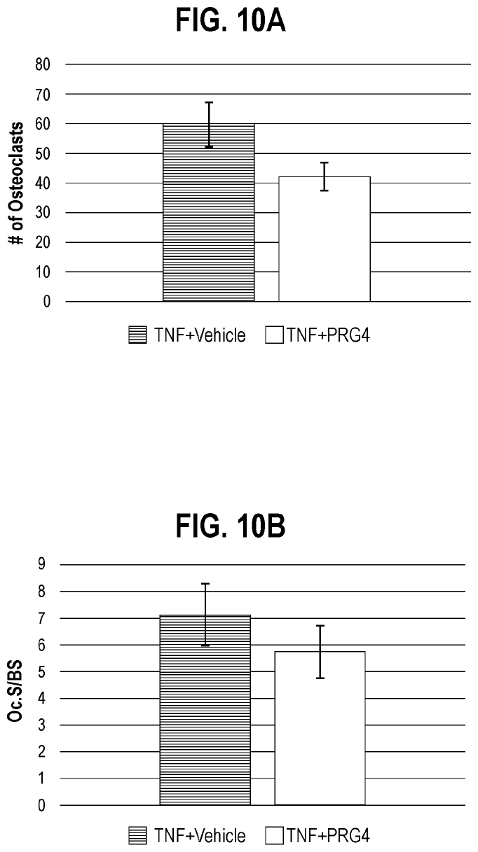

As shown in FIG. 10, in a preliminary analysis with a small sample size, there is indication that PRG4 may reduce osteoclast differentiation and osteoclast surface/bone surface in vivo. This is demonstrated by a slight reduction in number of osteoclasts (p=0.065, 1 tailed t test) and a potential reduction in osteoclast surface to bone surface (p=0.21, 1 tailed t test).

### Example 5: Mechanical Properties of PRG4 GT Mice+/−Tmx

In this experiment, the mechanical properties of PRG4 GT mice with and without tamoxifen (tmx) were studied. PRG4 GT mice lack global expression of the PRG4 gene (essentially, they are PRG4 KO mice) (9). However, endogenous expression can be “turned on” with injections of tamoxifen (tmx).

### Materials and Methods

The data described below was based on N=5 PRG4 GT mice in each group, all female, those that received tmx at 3 weeks of age (TMX+, 0.1 mg/gm body weight for 10 consecutive days) to re-express PRG4 and those that did not (TMX−). Mice were sacrificed at 2 months of age and the femurs were isolated, cleaned and subjected to 3-point bone testing using a Mach-1 Mechanical Tester Model v500csst. The opposite femur was prepared for μCT and evaluated using histomorphometric analysis as described previously (8).

Data was analyzed in combination with the geometry obtained from uCT (see Table 2 below) to calculate the mechanical properties of the bone (data is mean+/−SD).

### Results/Analysis

Re-expression of PRG4 resulted via tmx injections resulted in stiffer femurs (p=0.013), along with increased Young's modulus and ultimate stress (p=0.059 and 0.054 respectively, approaching significance) compared to bones deficient in PRG4.

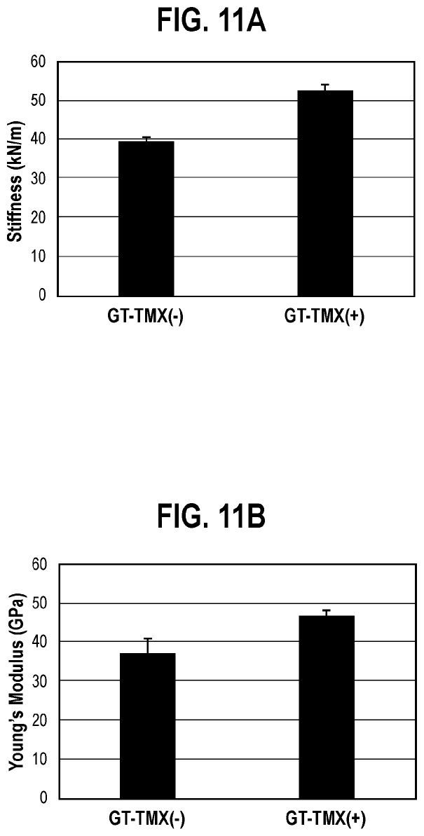

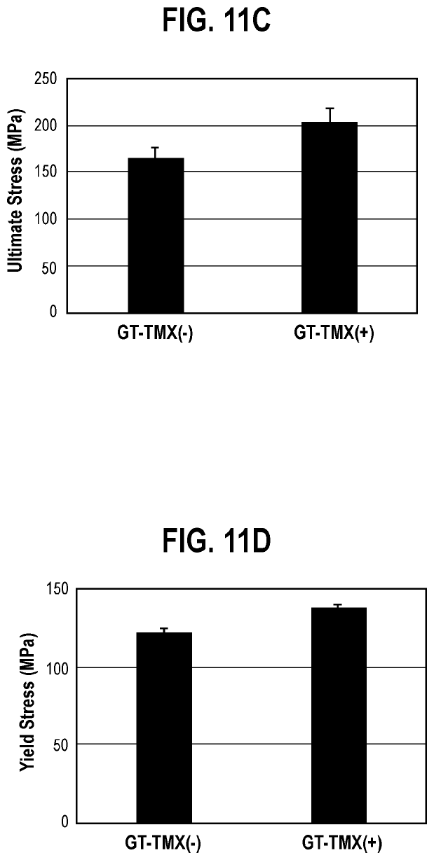

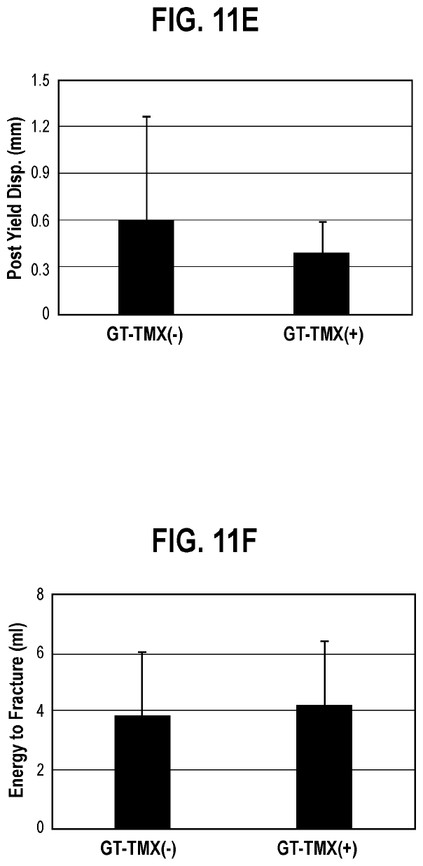

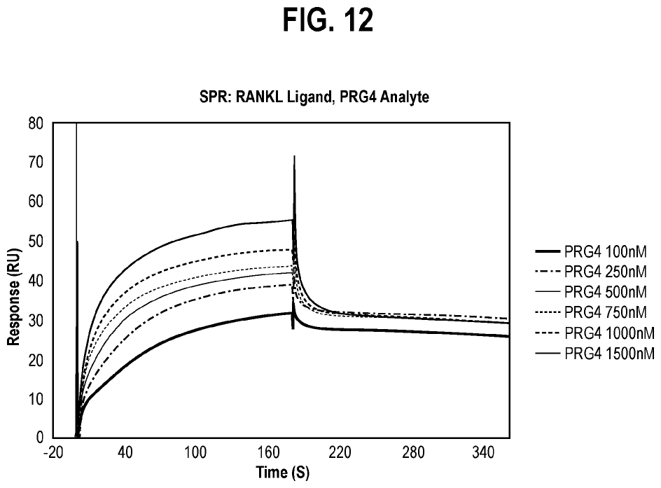

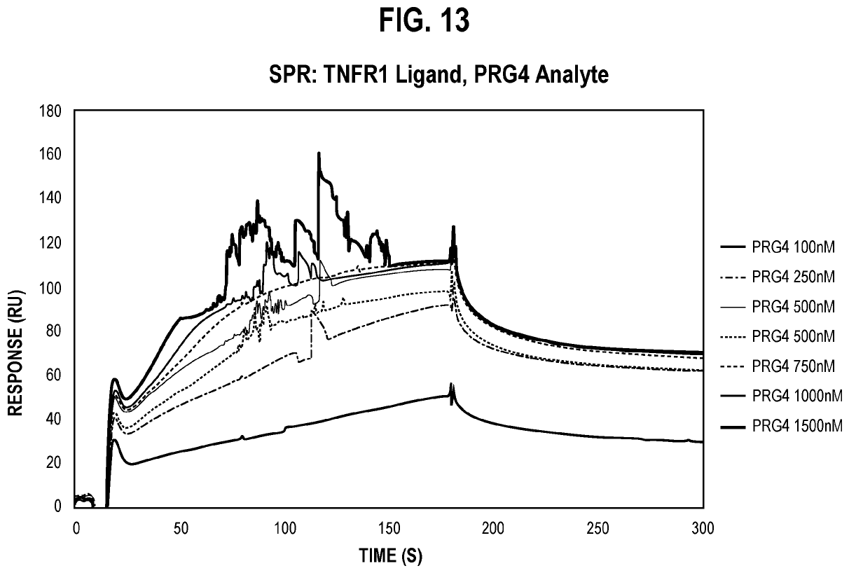

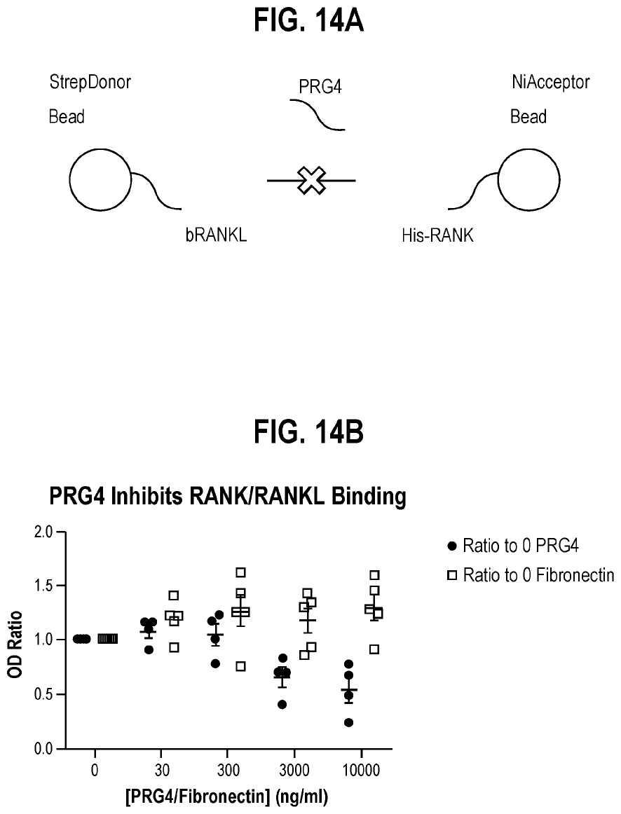

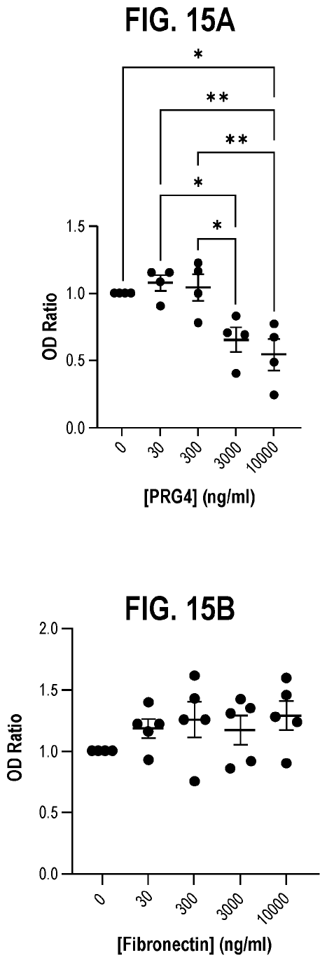

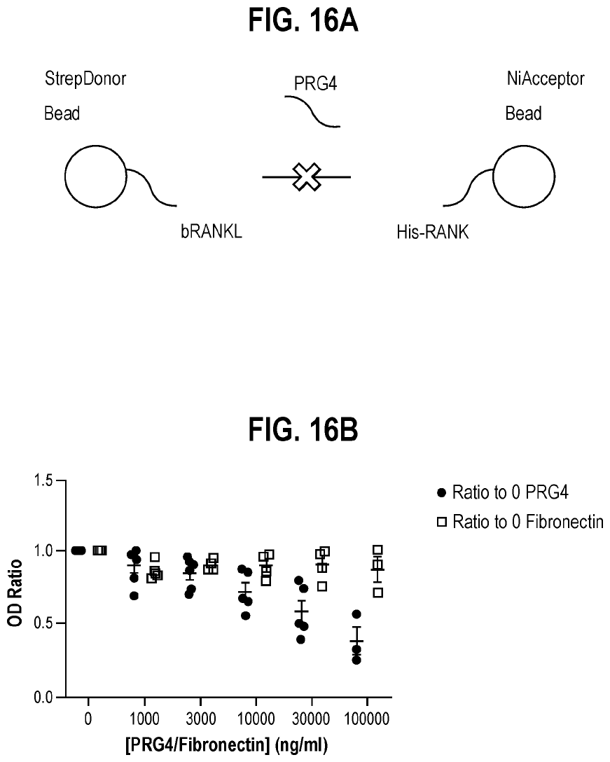

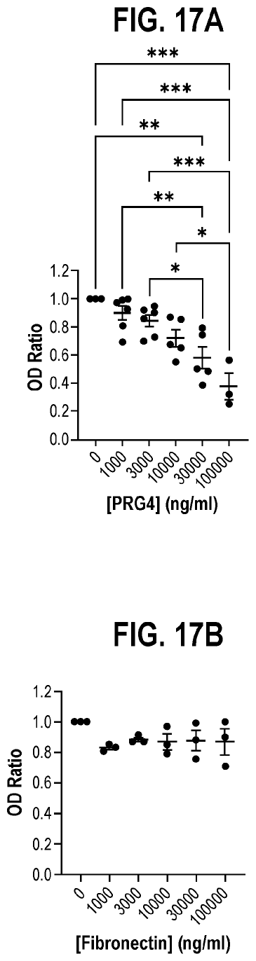

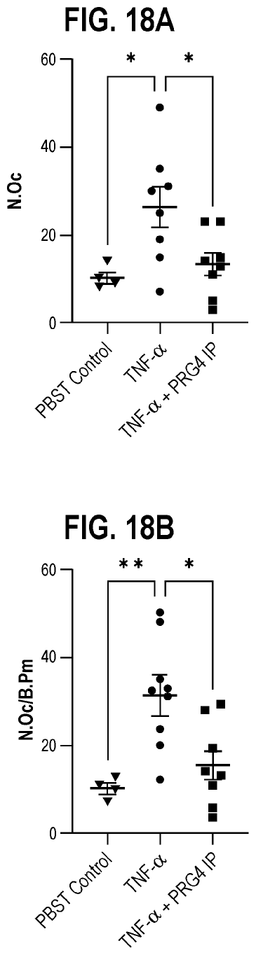

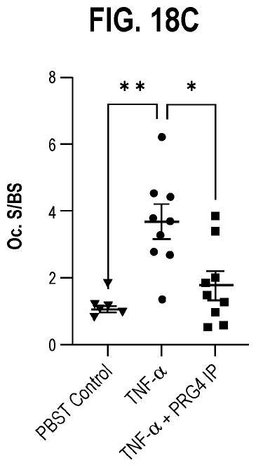

As shown in Table 1 and FIG. 11, these results demonstrate that lack of PRG4 expression results in diminished mechanical properties of bone, consistent with the notion of increased bone resorption in the absence of PRG4.

TABLE 1
Mechanical Properties of PRG4 GT mice +/− tmx

| | | Young's | Ultimate | Yield | Post Yield | Energy to |
|---|---|---|---|---|---|---|
| Mechanical | Stiffness | Modulus | Stress | Stress | Displacement | Fracture |
| Properties | (kN/m) | (GPa) | (MPa) | (MPa) | (mm) | (mJ) |
| GT-TMX(−) | 39.4 ± 2.7 | 7.36 ± 0.85 | 163.3 ± 12.1 | 122.4 ± 14.4 | 0.605 ± 0.661 | 3.8 ± 2.2 |
| GT-TMX(+) | 52.7 ± 3.2 | 9.34 ± 0.29 | 204.2 ± 13.6 | 138.5 ± 3.7 | 0.382 ± 0.186 | 4.2 ± 2.2 |
| P value | 0.013 | 0.059 | 0.054 | 0.309 | 0.488 | 0.750 |

Table 2 demonstrates mice deficient in endogenous PRG4 expression have altered bone micro architecture. After mechanical testing, femurs were prepared for uCT scanning. The trabecular and cortical bone properties are shown in Table 2.

The TMX-treated group had 37% more trabecular bone volume (p=0.029) and 32% more BV/TV (bone volume/total volume, approaching significance p=0.071). TMX treatment also resulted increased cortical bone area (p=0.039), 34% thicker cortical bone (p<0.00), along with decreased periosteal and endosteal perimeters (p<0.00). While tmx treatment itself can result in increased bone mass (10), these preliminary results suggest endogenous PRG4 (re)expression results in improved bone microarchitecture compared to bones deficient in PRG4.

TABLE 2
uCT Parameters in PRG4 GT Mice +/− tmx injection.

| | | | Trabecular | Trabecular | Trabecular | Connectivity | Structure |
|---|---|---|---|---|---|---|---|
| Trabecular | Bone Volume | | Thickness | Number | Spacing | Density | Model |
| Bone | (mm³) | Bv/tv (%) | (um) | (1/mm) | (um) | (1/mm³) | index |
| GT-TMX(−) | 0.215 ± 0.048 | 11.7 ± 3.8 | 35 ± 3 | 5.20 ± 0.52 | 188 ± 21 | 254 ± 50 | 2.2 ± 0.4 |
| GT-TMX(+) | 0.295 ± 0.047 | 15.4 ± 1.3 | 48 ± 18 | 5.30 ± 0.92 | 191 ± 57 | 289 ± 75 | 2.1 ± 0.1 |
| P value | 0.029 | 0.071 | 0.166 | 0.836 | 0.905 | 0.410 | 0.450 |

| | | | Cortical | Periosteal | Endosteal |
|---|---|---|---|---|---|
| Cortical | Cortical Bone | Cortical | Thickness | Perimeter | Perimeter |
| Bone | Area (mm2) | Porosity (%) | (mm) | (mm) | (mm) |
| GT-TMX(−) | 0.429 ± 0.047 | 0.95 ± 0.25 | 0.11 ± 0.01 | 5.73 ± 0.09 | 3.14 ± 0.09 |
| GT-TMX(+) | 0.498 ± 0.042 | 0.73 ± 0.14 | 0.15 ± 0.01 | 4.99 ± 0.27 | 2.61 ± 0.13 |
| P value | 0.039 | 0.114 | 0.000 | 0.000 | 0.000 |

This data is in general consistent with previous data showing PRG4 KO mice have decreased trabecular and cortical bone compared to WT (11), a study motivated in part by previous observations of osteopenia in PRG4 KO mice (12) and the fact PRG4 is endogenously expressed in bone (13).

### Example 6: Measuring Strength of Binding Between RANKL and rhPRG4

Surface plasmon resonance (SPR) was performed using a Biacore T200 (GE Healthcare Life Sciences, Marlborough, Mass.). Biotinylated-RANKL (Acro Biosystems, Newark, Del.) was flowed at 2000 nM in 10 mM Sodium Acetate buffer (pH 4.5) with a flow rate of 20 ul/min for 20 minutes and immobilized on a CM5 Series S chip, producing a signal of 2100 RU due to immobilization. Next, rhPRG4 was flowed over the chip at 0, 100, 250, 500(×2), 750, 1000, and 1500 nM concentrations in 10 mM Sodium Hepes (pH 7.4, 175 mM NaCl, 0.05% Polysorbate 20) with a flow rate of 30 ul/min, contact time of 180 s, and dissociation time of 180 s. Bound rhPRG4 was removed using 100 mM glycine pH 2.0 at a flow rate of 50 ul/min with a contact time of 30 s. The dissociation constant was calculated using Biacore T200 Evaluation software and fitted using 1:1 binding.

### Example 7: Measuring Strength of Binding Between TNFR1 and rhPRG4

Surface plasmon resonance (SPR) was performed using a Biacore T200 (GE Healthcare Life Sciences, Marlborough, Mass.). His-tagged TNFR1 (Acro Biosystems, Newark, Del.) was flowed at 2000 nM in 10 mM Sodium Acetate buffer (pH 4.5) with a flow rate of 15 ul/min for 18 minutes and immobilized on a CM5 Series S chip, producing a signal of 2641 RU due to immobilization. Next, rhPRG4 was flowed over the chip at 0, 100, 250, 500(×2), 750, 1000, and 1500 nM concentrations in 10 mM Sodium Hepes (pH 7.4, 175 mM NaCl, 0.05% Polysorbate 20) with a flow rate of 30 ul/min, contact time of 180 s, and dissociation time of 180 s. Bound rhPRG4 was removed using 100 mM glycine pH 2.0 at a flow rate of 50 ul/min with a contact time of 30 s. The dissociation constant was calculated using Biacore T200 Evaluation software and fitted using 1:1 binding.

All patents, publications and abstracts cited above are incorporated herein by reference in their entirety. It should be understood that the foregoing relates only to preferred embodiments of the present invention and that numerous modifications or alteration may be made therein without departing from the spirit and the scope of the present invention as defined in the following claims.

### REFERENCES

1. Khosla S, Shane E. A Crisis in the Treatment of Osteoporosis. J Bone Miner Res. 2016; 31(8):1485-7. Epub 2016/06/24. doi: 10.1002/jbmr.2888. PubMed PMID: 27335158.
2. Khosla S, Burr D, Cauley J, Dempster D W, Ebeling P R, Felsenberg D, Gagel R F, Gilsanz V, Guise T, Koka S, McCauley L K, McGowan J, McKee M D, Mohla S, Pendrys D G, Raisz L G, Ruggiero S L, Shafer D M, Shum L, Silverman S L, Van Poznak C H, Watts N, Woo S B, Shane E. Bisphosphonate-associated osteonecrosis of the jaw: report of a task force of the American Society for Bone and Mineral Research. J Bone Miner Res. 2007; 22(10):1479-91. PubMed PMID: 17663640.
3. Shane E, Burr D, Ebeling P R, Abrahamsen B, Adler R A, Brown T D, Cheung A M, Cosman F, Curtis J R, Dell R, Dempster D, Einhom T A, Genant H K, Geusens P, Klaushofer K, Koval K, Lane J M, McKiernan F, McKinney R, Ng A, Nieves J, O'Keefe R, Papapoulos S, Sen H T, van der Meulen M C, Weinstein R S, Whyte M. Atypical subtrochanteric and diaphyseal femoral fractures: Report of a task force of the American Society for Bone and Mineral Research. J Bone Miner Res. 2010; 14:14. PubMed PMID: 20842676.
4. Quattrocchi E, Kourlas H. Teriparatide: a review. Clin Ther. 2004; 26(6):841-54.

Epub 2004/07/21. PubMed PMID: 15262455.
5. O'Donoghue M, Cavallari I, Bonaca M, Wiviott S, Cange A, Laura G, Deenadayalu N, Im K, Murphy S, Sabatine M, editors. The Long-Term Odanacatib Fracture Trial (LOFT): Cardiovascular Safety Results. The Long-Term Odanacatib Fracture Trial (LOFT): Cardiovascular Safety Results, Abstract #1155, Annual Meeting, American Society for Bone and Mineral Research; 2016; Atlanta, Ga.
6. Lewiecki E M, Blicharski T, Goemaere S, Lippuner K, Meisner P D, Miller P D, Miyauchi A, Maddox J, Chen L, Horlait S. A Phase III Randomized Placebo-Controlled Trial to Evaluate Efficacy and Safety of Romosozumab in Men With Osteoporosis. J Clin Endocrinol Metab. 2018; 103(9):3183-93. Epub 2018/06/23. doi: 10.1210/jc.2017-02163. PubMed PMID: 29931216.
7. Miller P D, Hattersley G, Riis B J, Williams G C, Lau E, Russo L A, Alexandersen P, Zerbini C A, Hu M Y, Harris A G, Fitzpatrick L A, Cosman F, Christiansen C. Effect of Abaloparatide vs Placebo on New Vertebral Fractures in Postmenopausal Women With Osteoporosis: A Randomized Clinical Trial. JAMA. 2016; 316(7):722-33. Epub 2016/08/18. doi: 10.1001/jama.2016.11136. PubMed PMID: 27533157.
8. Jastrzebski S, Kalinowski J, Mun S, Shin B, Adapala N S, Jacome-Galarza C E, Mirza F, Aguila H L, Drissi H, Sanjay A, Canalis E, Lee S K, Lorenzo J A. Protease-Activated Receptor 1 Deletion Causes Enhanced Osteoclastogenesis in Response to Inflammatory Signals through a Notch2-Dependent Mechanism. J Immunol. 2019; 203(1):105-16. Epub 2019/05/22. doi: 10.4049/jimmunol.1801032. PubMed PMID: 31109956; PMCID: PMC6581625.
9. Hill A, Waller K A, Cui Y, Allen J M, Smits P, Zhang L X, Ayturk U M, Hann S, Lessard S G, Zurakowski D, Warman M L, Jay G D. Lubricin restoration in a mouse model of congenital deficiency. Arthritis Rheumatol. 2015; 67(11):3070-81. doi: 10.1002/art.39276. PubMed PMID: 26216721; PMCID: PMC4626303.
10. Starnes L M, Downey C M, Boyd S K, Jirik F R. Increased bone mass in male and female mice following tamoxifen administration. Genesis. 2007; 45(4):229-35. Epub 2007/04/10. doi: 10.1002/dvg.20294. PubMed PMID: 17417806.
11. Novince C M, Michalski M N, Koh A J, Sinder B P, Entezami P, Eber M R, Pettway G J, Rosol T J, Wronski T J, Kozloff K M, McCauley L K. Proteoglycan 4: a dynamic regulator of skeletogenesis and parathyroid hormone skeletal anabolism. J Bone Miner Res. 2012; 27(1):11-25. Epub 2011/09/21. doi: 10.1002/jbmr.508. PubMed PMID: 21932346; PMCID: PMC4118835.
12. Rhee D K, Marcelino J, Baker M, Gong Y, Smits P, Lefebvre V, Jay G D, Stewart M, Wang H, Warman M L, Carpten J D. The secreted glycoprotein lubricin protects cartilage surfaces and inhibits synovial cell overgrowth. J Clin Invest. 2005; 115(3):622-31. PubMed PMID: 15719068.
13. Ikegawa S, Sano M, Koshizuka Y, Nakamura Y. Isolation, characterization and mapping of the mouse and human PRG4 (proteoglycan 4) genes. Cytogenet Cell Genet. 2000; 90:291-7.
14. Osteoporosis Canada website: osteoporosis.ca/about-the-disease/what-is-osteoporosis/secondary-osteoporosis/medical-conditions-that-can-cause-bone-loss-falls-and/or-fractures/.
15. Steele, B. L., Alvarez-Veronesi, M. C. and Schmidt, T. A., Molecular Weight Characterization of PRG4 Proteins Using Multi-Angle Laser Light Scattering (MALLS) Osteoarthritis Cartilage 2013 March; 21(3):498-504. doi: 10.1016/j.joca.2012.12.002. Epub 2012 Dec. 17. PubMed PMID: PMID 23257245.

1. A method for reducing or inhibiting bone resorption comprising administering to a human or animal an anti-osteoclastic composition comprising proteoglycan 4 and a pharmaceutically acceptable carrier.

2. The method of claim 1, wherein the proteoglycan 4 is recombinant human proteoglycan 4.

3. The method of claim 1, wherein the pharmaceutically acceptable carrier is a hydrogel or DNA-inspired nanomaterial.

4. The method of claim 3, wherein the anti-osteoclastic composition is administered directly to the area of the human or animal that is experiencing or susceptible to bone resorption.

5. The method of claim 4, wherein the area of the human or animal that is experiencing or susceptible to bone resorption is a bone.

6. The method of claim 1, wherein administration of the anti-osteoclastic composition inhibits or delays onset of a pathological condition of the bone.

7. The method of claim 6, wherein the pathological condition of the bone is osteopenia.

8. The method of claim 6, wherein the pathological condition of the bone is osteoporosis.

9. The method of claim 6, wherein the pathological condition of the bone is bone loss or resorption associated with cancer.

10. The method of claim 1 further comprising administration of a second anti-osteoclastic composition.

11. An anti-osteoclastic composition comprising a proteoglycan 4 and a pharmaceutically acceptable carrier, wherein the pharmaceutically acceptable carrier is a hydrogel or DNA-inspired nanomaterial.

12. An anti-osteoclastic composition comprising a proteoglycan 4 and a pharmaceutically acceptable carrier in combination with a second anti-osteoclastic composition.

13. The composition of claim 11, wherein the proteoglycan 4 is recombinant human proteoglycan 4.

14. The composition of claim 12, wherein the proteoglycan 4 is recombinant human proteoglycan 4.
